(19) United States
(12) Patent Application Publication
(10) Pub. No.: US 2007/0288005 A1
(43) Pub. Date: Dec. 13, 2007

(54) FIXATION OF SPINAL PROSTHESIS

(76) Inventors: Uri Arnin, Kiryat Tivon (IL); Shai Fleischer, Haifa (IL)

Correspondence Address: David Klein;DEKEL PATENT LTD., Beit HaRof'im, 18 Menuha VeNahala Street, Room 27, REHOVOT, omitted

(21) Appl. No.: 11/692,195

(22) Filed: Mar. 28, 2007

Related U.S. Application Data
(60) Provisional application No. 60/789,220, filed on Apr. 5, 2006.
(60) Provisional application No. 60/849,447, filed on Oct. 5, 2006.

(51) Int. Cl. A61B 17/58

(52) U.S. Cl. 606/61; 606/72

(57) ABSTRACT

An endoprosthesis including first and second attachment surfaces for attachment to spinal structure, and a fixation element rotatingly disposed in at least one of the attachment surfaces, the fixation element being rotatable between a stowed position, wherein the fixation element does not substantially protrude from the at least one of the attachment surfaces, and a deployed position, wherein the fixation element does substantially protrude from the at least one of the attachment surfaces for entering and being fixed to the spinal structure.

### CROSS-REFERENCE TO RELATED APPLICATIONS

This application claims priority under 35 USC §119 to U.S. Provisional Patent Application Ser. No. 60/789,220, filed on Apr. 5, 2006, and 60/849,447, filed Oct. 5, 2006, which are incorporated herein by reference.

### FIELD OF THE INVENTION

The present invention relates to spinal prostheses and particularly fixation of prostheses for disc or partial spinal disc replacement.

### BACKGROUND OF THE INVENTION

Spinal disc or partial spinal disc replacement is a common procedure with thousands having been performed already and. Examples of such replacement discs are ProDisc (Spine Solutions, Inc.) and SB Charite III (Link Spine Group, Inc.)

By replacing a disc and not merely fusing vertebrae, which is the common alternative practice to disc replacement, the mobility of the patient's adjacent discs may be preserved and thus may delay the onset of arthritic changes to adjacent vertebrae.

Although many prosthetic disc devices are described in the literature, there is still a need for improvement in ease of performance

Artificial discs usually have spikes, keels, screws or other protrusions attached to or integrated into the outer surfaces of these prostheses in order to assist in the initial fixation to the endplates of the treated vertebrae that help in the prevention of migration of the prostheses away from their correct positioning in the intravertebral space.

When a disc is to be placed into the intravertebral space, a space needs to be prepared that will take into account the total height of the prosthesis, including the added height of the protrusions. A solution is required that would not necessitate a distraction of the added height of the protrusions.

### SUMMARY OF THE INVENTION

The present invention is directed to a spinal prosthesis, as is described more in detail herein below. Attachments members or fixation elements, such as spikes, ridges, keels, teeth, pegs, and fins and any other extrusions or protrusions (all these terms being used interchangeably throughout the specification and claims), initially do not significantly protrude above the external end plates of the prosthesis. The fixation elements are deployed to a position that extends or protrudes beyond the exterior surface of the endoprosthesis only after inserting the endoprosthesis (or prosthesis or implant, all these terms being used interchangeably throughout the specification and claims) into the inter-vertebral space. Only when the prosthesis is in its correct final position, the protrusions are manipulated into position for fixation. The initial low-profile of the implant reduces the amount of distraction needed and avoids over-distraction or chiseling of the vertebrae.

### BRIEF DESCRIPTION OF THE DRAWINGS

In the drawings:

FIGS. 1A and 1B are simplified illustrations of a vertebral disc endoprosthesis, constructed and operative in accordance with an embodiment of the present invention, having fixation elements that are press-fit into channels;

FIGS. 2A and 2B are simplified illustrations of a vertebral disc endoprosthesis, constructed and operative in accordance with an embodiment of the present invention, having fixation elements that are screwed into channels;

FIGS. 3A and 3B are simplified illustrations of a vertebral disc endoprosthesis, constructed and operative in accordance with another embodiment of the present invention, having fixation elements that are press-fit into channels and have a dove tail;

FIGS. 4A and 4B are simplified illustrations of a vertebral disc endoprosthesis, constructed and operative in accordance with another embodiment of the present invention, having fixation elements that are press-fit into channels and are formed with a trapezoidal backing;

FIGS. 5A and 5B are simplified illustrations of a vertebral disc endoprosthesis, constructed and operative in accordance with another embodiment of the present invention, having fixation elements that are press-fit into channels, have a trapezoidal backing and a protruding part in the form of a dove tail;

FIGS. 6A-6C are simplified illustrations of a vertebral disc endoprosthesis, constructed and operative in accordance with another embodiment of the present invention, having fixation elements that comprise pins inserted in sharp angle holes;

FIGS. 7A-7C are simplified illustrations of a vertebral disc endoprosthesis, constructed and operative in accordance with another embodiment of the present invention, having fixation elements that comprise pins that are curved;

FIGS. 8A-8C and FIGS. 9A-9C are simplified illustrations of a vertebral disc endoprosthesis, constructed and operative in accordance with another embodiment of the present invention, having fixation elements that are rotatingly disposed in channels, in respective stowed and deployed positions, wherein the fixation elements have fixing members that terminate in a dove tail;

FIGS. 10A-10C and FIGS. 11A-11C are simplified illustrations of a vertebral disc endoprosthesis, constructed and operative in accordance with another embodiment of the present invention, having fixation elements that are rotatingly disposed in channels, in respective stowed and deployed positions, wherein the fixation elements have fixing members that terminate in an elongate sharp or narrow edge;

FIGS. 12A-12C and FIGS. 13A-13D are simplified illustrations of a vertebral disc endoprosthesis, constructed and operative in accordance with another embodiment of the present invention, having fixation elements that are rotatingly disposed in channels, in respective stowed and deployed positions, wherein the fixation elements pivot about a pivot pin;

FIGS. 14A-14C and FIGS. 15A-15C are simplified illustrations of a vertebral disc endoprosthesis, constructed and operative in accordance with another embodiment of the present invention, having fixation elements that are rotatingly disposed in channels, in respective stowed and deployed positions, wherein the fixation elements have fixing members that terminate in an elongate sharp or narrow edge;

FIGS. 16A-16C are simplified illustrations of a variant of the embodiment of FIGS. 14A-15C, which geometrically locks the fixing member of the fixation element in place;

FIGS. 17A-17C are simplified illustrations of a vertebral disc endoprosthesis, constructed and operative in accordance with another embodiment of the present invention, having fixation elements with a plurality of fixing members formed thereon;

FIG. 18 is a simplified illustration of a surgical procedure for manipulating the fixation elements, in accordance with an embodiment of the present invention; and

FIGS. 19A-19D, 20A-20D, 21A-21C and 22 are simplified illustrations of fixation elements, constructed and operative in accordance with different embodiments of the present invention, wherein FIGS. 19A and 19B illustrate a fixation element that has a spike, FIGS. 19C and 19D illustrate other variants of the fixation element, respectively with two and three spikes, FIGS. 20A and 20B illustrate a fixation element manufactured from more than one part, FIGS. 20C and 20D illustrate a fixation element that includes locking members for locking the fixation element in the stowed and deployed positions, FIGS. 21A and 21B illustrate another kind of locking member for locking the fixation element in the stowed and deployed positions in the channel, FIG. 21C illustrates another kind of locking member for locking the fixation element in the stowed and deployed positions, and FIG. 22 the fixation element formed with a hole in the spike.

### DESCRIPTION OF EMBODIMENTS

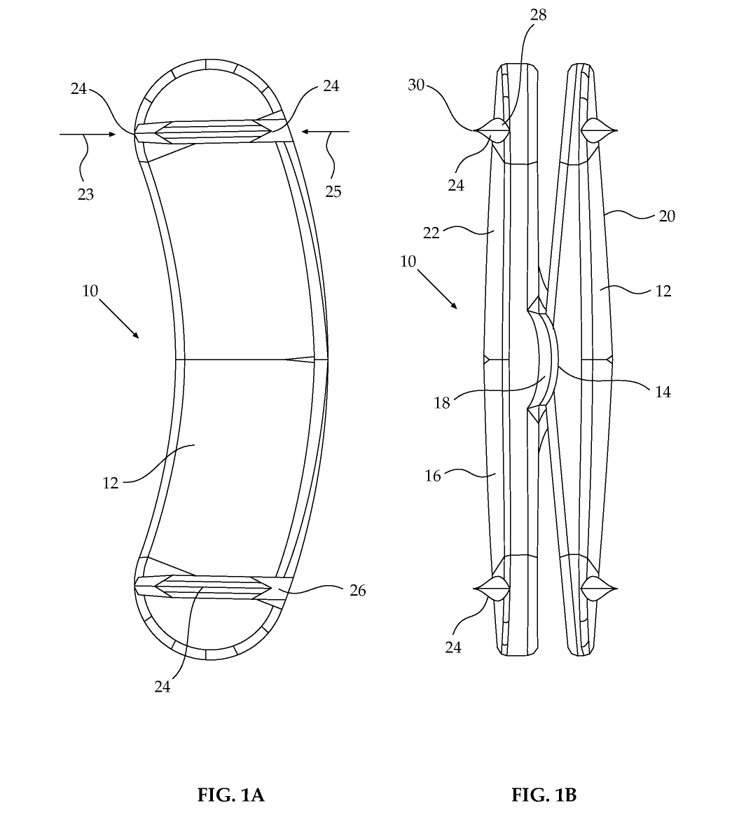

Reference is now made to FIGS. 1A and 1B, which illustrate a vertebral disc endoprosthesis 10, constructed and operative in accordance with an embodiment of the present invention. Endoprosthesis 10 may be a spinal endoprosthesis for disc or partial spinal disc replacement, similar to that described in PCT published patent application WO 2005/084590, the disclosure of which is incorporated herein by reference.

Endoprosthesis 10 may be fashioned from any combination of materials used for spinal prostheses, such as but not limited to, ceramic, aluminum oxide, zirconium oxide, metal, metal alloy, cobalt-chromium-tungsten-nickel alloy, cobalt-chromium-manganese alloy, cobalt-chromium alloy, cobalt-chromium-molybdenum alloy, chromium-nickel-manganese alloy, powder metal alloy, 316L stainless steel, Ti 6Al-4V ELI (nominally 6 percent aluminum, 4 percent vanadium, balance by weight titanium, extra-low interstitial grade), polymer, polyurethane, polyethylene, wear resistant polyethylene, cross-linked polyethylene, thermoplastic elastomer, biomaterial, polycaprolactone, diffusion hardened material, Ti-13-13, zirconium alloy, niobium alloy, with or without a porous coating system, hydrophilic coating system, hydroxyapatite coating system, and tri-calcium phosphate coating system.

In accordance with a non-limiting embodiment of the invention, endoprosthesis 10 may include a first articulating member 12 formed with a generally concave articulating surface 14, and a second articulating member 16 formed with a generally convex articulating surface 18 which articulates with concave articulating surface 14. The first and second articulating members 12 and 16 may have generally planar attachment surfaces 20 and 22, respectively, for attachment to structure of the spine. The attachment surface 20 may be generally flat, but can be curved or irregular, depending on the application.

In accordance with a non-limiting embodiment of the invention, attachment surfaces 20 and 22 include fixation elements (e.g., spikes or other protrusions) 24 to improve the attachment to the spine structure.

It may be disadvantageous to have fixation elements 24 in place on attachment surfaces 20 and 22 before the endoprosthesis 10 is inserted. For example, if the fixation elements 24 were to initially protrude from attachment surfaces 20 and 22, it may be difficult to insert endoprosthesis 10 into the intervertebral space and position it in the recommended/preferred/best position. The fixation elements 24 could interfere with insertion because the spikes could catch or snag on spinal structure prior to reaching the final desired position of the endoprosthesis. An additional disadvantage is that if the fixation elements 24 were to initially protrude from attachment surfaces 20 and 22 the endoprosthesis total height would be slightly higher. The added height could make initial placement of the endoprosthesis more difficult.

In accordance with a non-limiting embodiment of the invention, attachment surfaces 20 and 22 are formed with one or more channels 26 and fixation elements 24 are inserted into channels 26 only after endoprosthesis 10 into the intervertebral space. In the embodiment of FIGS. 1A and 1B, fixation elements 24 are press-fit into channels 26 by pushing them into channels 26 in either the direction of arrow 23 or 25 (whichever is more convenient). The fixation elements 24 of this embodiment are formed with an arcuate backing 28 (in which case channel 26 may be arcuate as well) with an elongate sharp edge 30. The elongate sharp edge 30 digs into the spinal structure upon insertion of fixation elements 24 into channel 26 and fixes the endoprosthesis 10 into the intervertebral space.

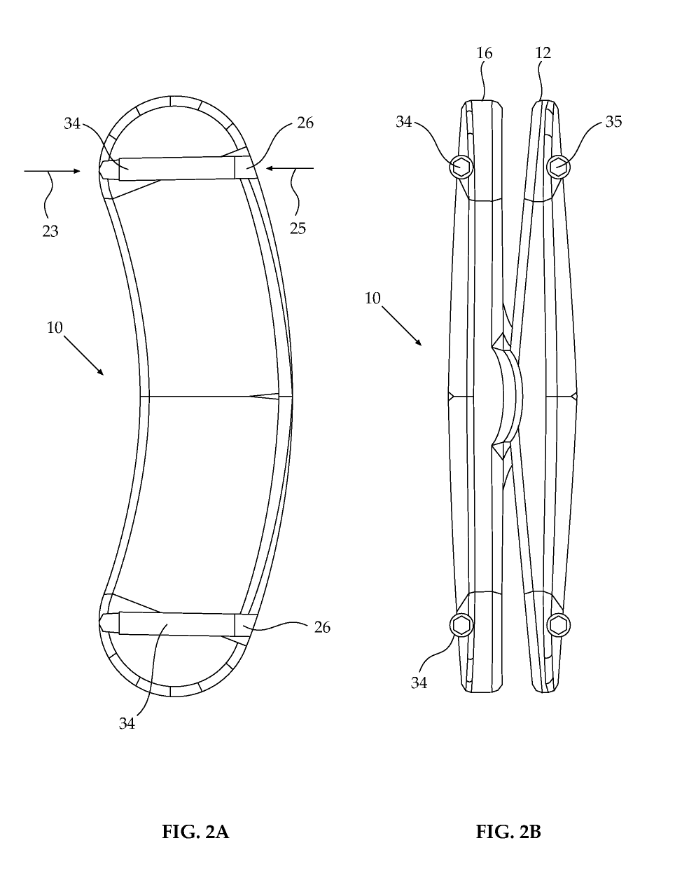

In the embodiment of FIGS. 2A and 2B, fixation elements 34 are provided, which are formed as a thread rod or bar and are screwed into channels 26 by screwing in either the direction of arrow 23 or 25 (whichever is more convenient). The fixation elements 34 may be self-tapping and screw into the spinal structure to fix the endoprosthesis 10 into the intervertebral space. Channels 26 may be tapped or may be previously un-tapped, in which case fixation elements 34 may form threads in channels 26 as they are screwed in place. The fixation element 34 may have a driver interface 35, such as a regular screwdriver slot, Philips screwdriver slot or hexagonal Allen socket (the latter being in the illustrated embodiment), for screwing by means of a suitable tool.

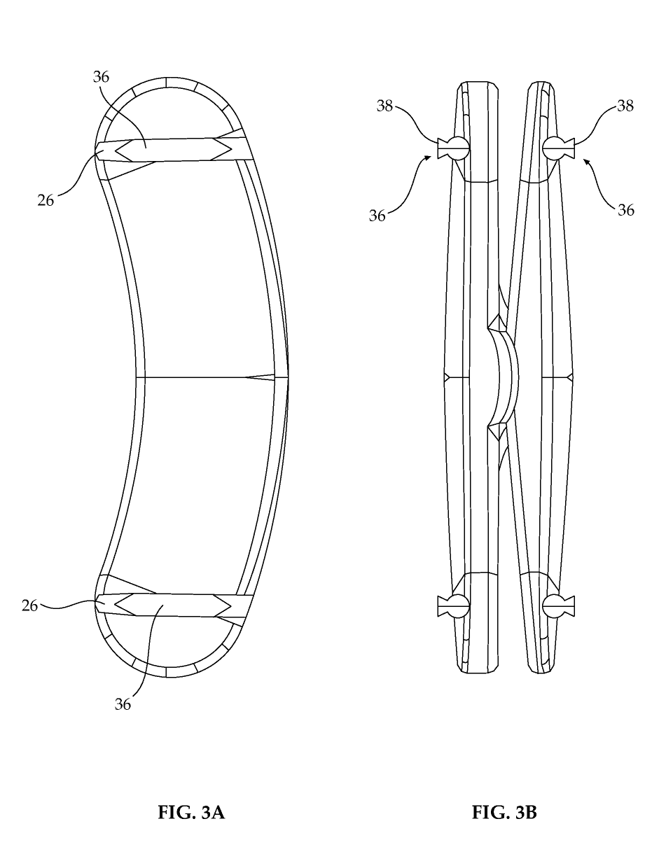

In the embodiment of FIGS. 3A and 3B, fixation elements 36 are press-fit into channels 26 and the protruding part is in the form of a dove tail 38.

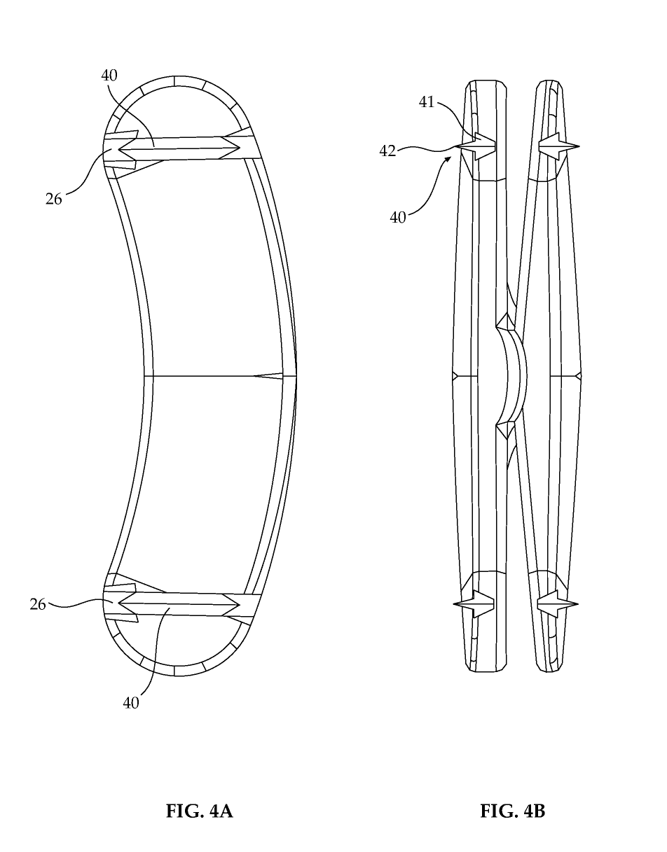

In the embodiment of FIGS. 4A and 4B, fixation elements 40 are press-fit into channels 26. Fixation elements 40 are formed with a trapezoidal backing 41 (in which case channel 26 may be complementary trapezoidal) with an elongate sharp edge 42.

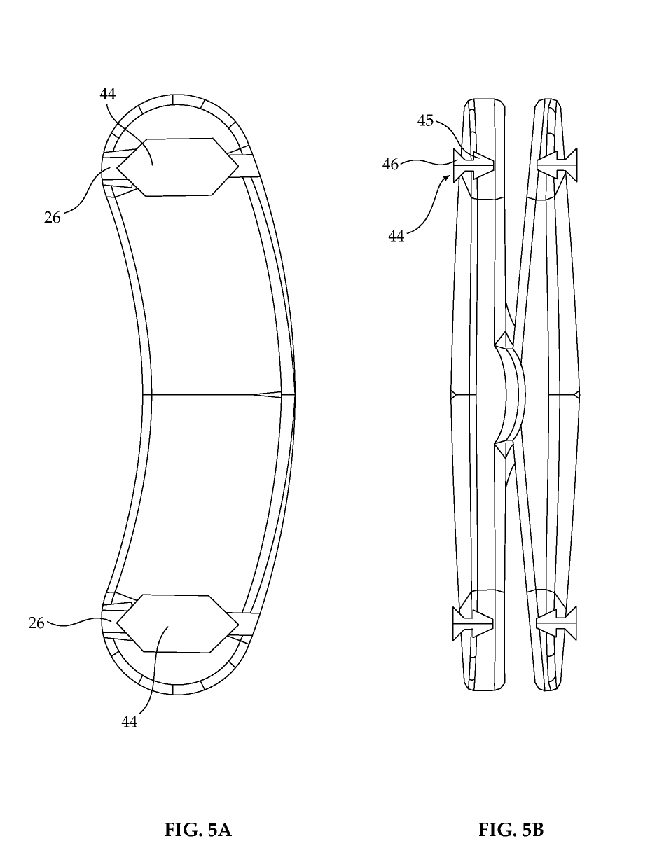

In the embodiment of FIGS. 5A and 5B, fixation elements 44 are press-fit into channels 26. Fixation elements 44 are formed with a trapezoidal backing 45 (in which case channel 26 may be complementary trapezoidal) and the protruding part is in the form of a dove tail 46.

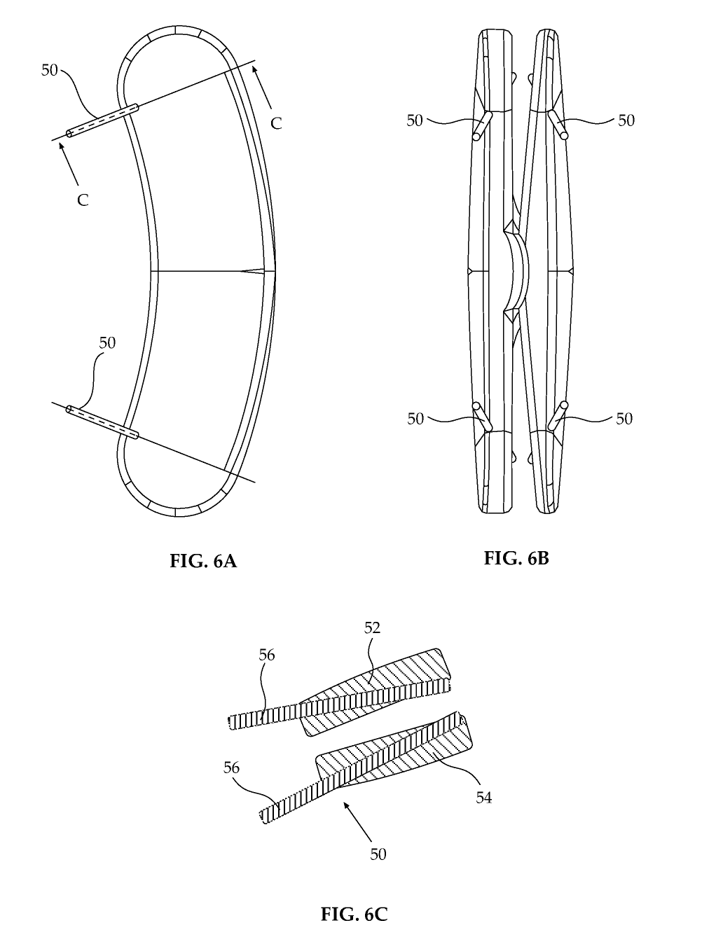

In the embodiment of FIGS. 6A and 6B, fixation elements 50 comprise pins inserted in sharp angle holes 52 or 54 into the vertebra. The pins may have protrusions 56 when in place, as shown in FIG. 6C.

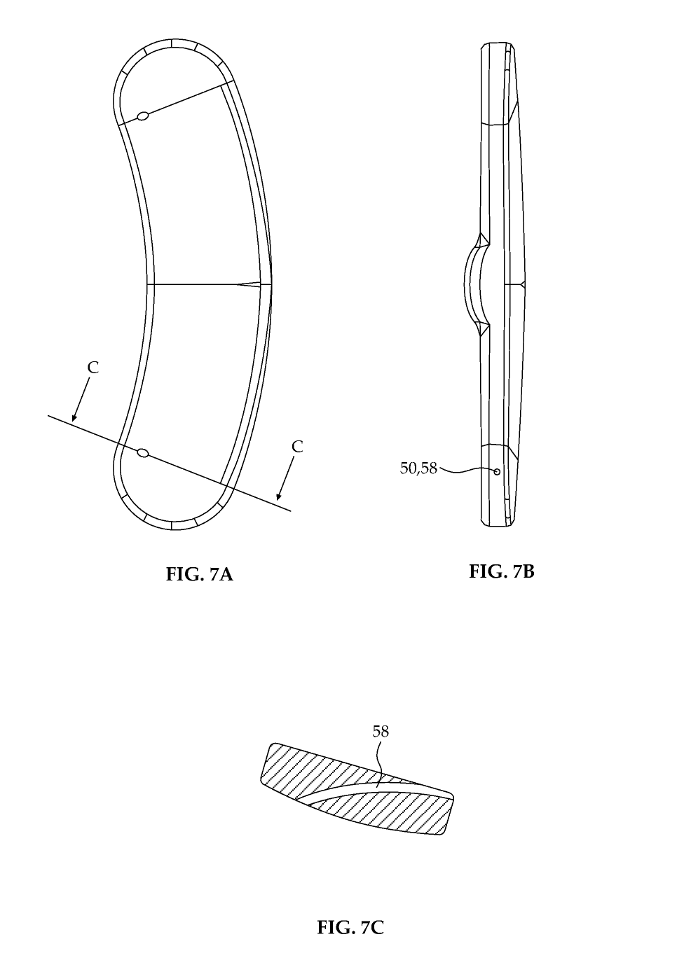

In the embodiment of FIGS. 7A and 7B, the fixation elements are the same as that of FIGS. 6A and 6B, except that the pins are curved (bent or plastic deformation and the like) and fit in a curved track 58 (FIG. 7C) formed in the prosthesis.

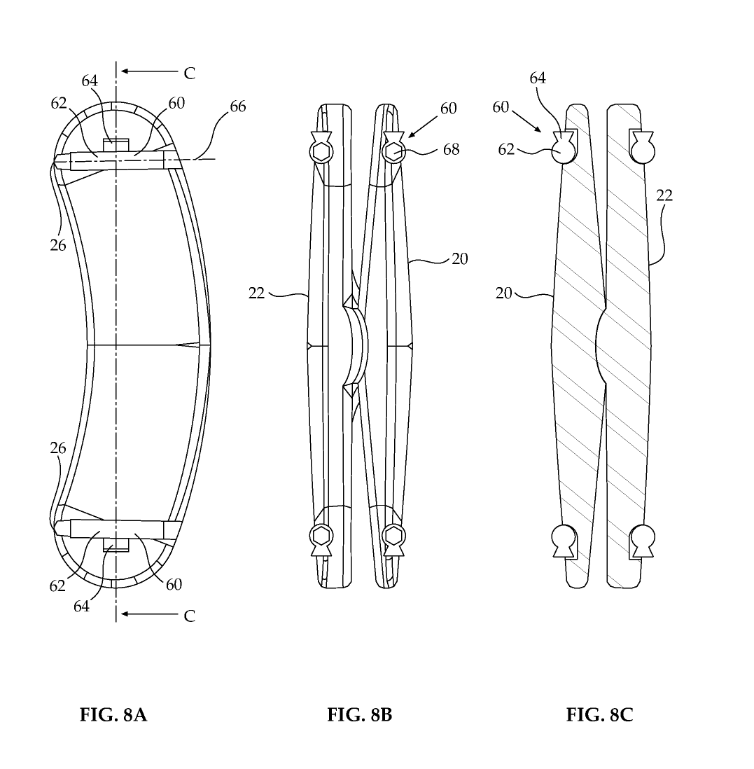

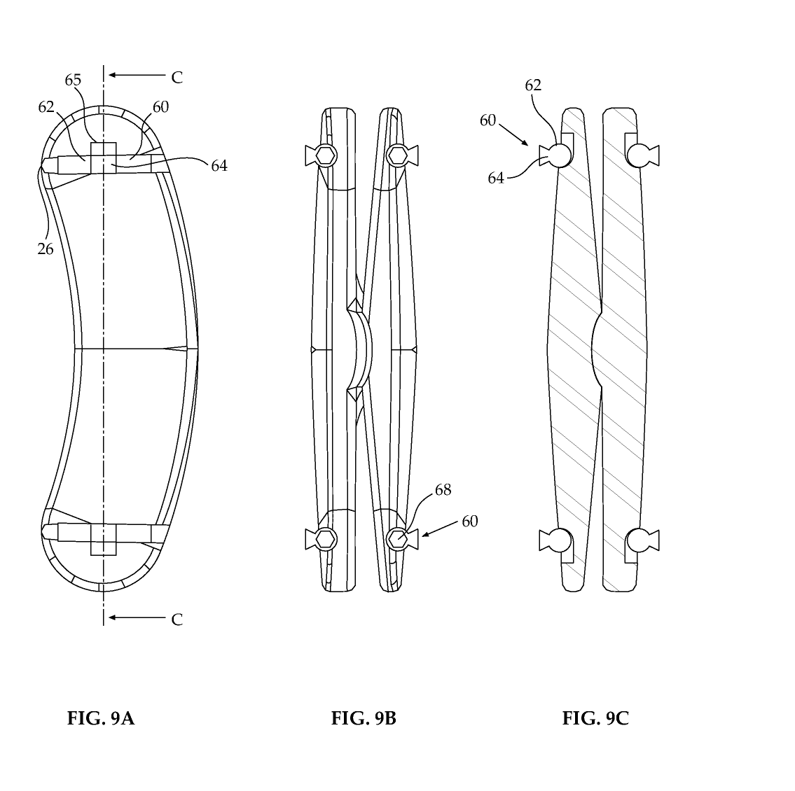

Reference is now made to FIGS. 8A-8C. In accordance with a non-limiting embodiment of the invention, attachment surfaces 20 and 22 are formed with one or more channels 26 and fixation elements 60 are rotatingly disposed in channels 26. In the embodiment of FIGS. 8A-8C, fixation elements 60 are formed as a rod or bar 62 with a fixing member 64 that protrudes from rod 62. Fixing member 64 (which in the illustrated embodiment is in the form of a dove tail) may initially sit in a groove 65 (seen best in FIG. 9A), which extends from channel 26. The fixation element 60 can rotate about a longitudinal axis 66 of rod 62. The fixation element 60 may have a driver interface 68, such as a regular screwdriver slot, Philips screwdriver slot or hexagonal Allen socket (the latter being in the illustrated embodiment), for turning by means of a suitable tool.

Initially, fixing member 64 sits in groove 65. Upon rotation of fixation element 60, fixing member 64 turns out of groove 65 and is deployed to the position shown in FIGS. 9A-9C to press or “bite” into the spinal structure and fix the endoprosthesis into the intervertebral space.

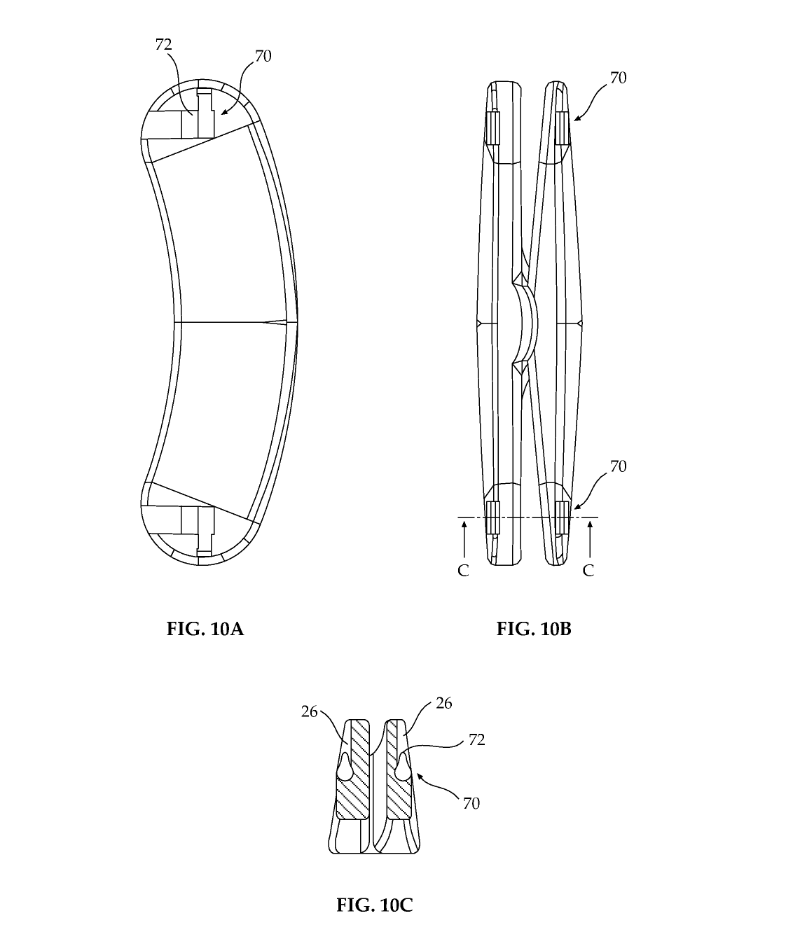

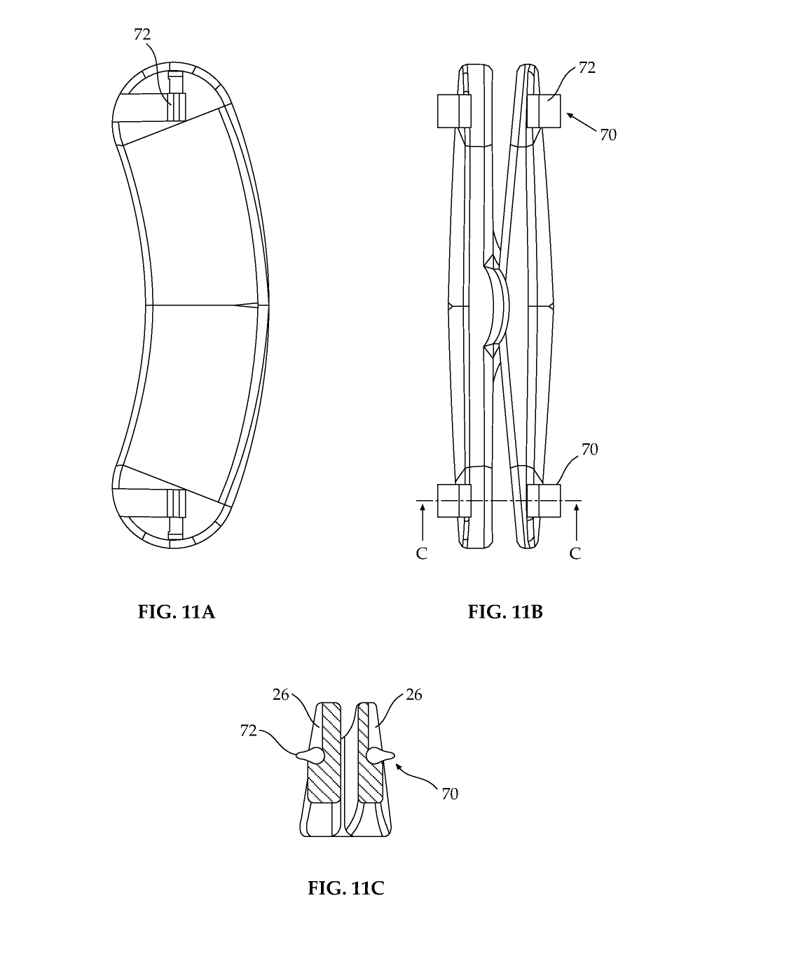

Reference is now made to FIGS. 10A-11C. This embodiment is basically the same as that of FIGS. 8A-9C, except that in the embodiment of FIGS. 10A-11C, there are fixation elements 70 that have fixing members 72 that terminate in an elongate sharp or narrow edge instead of terminating in a dove tail.

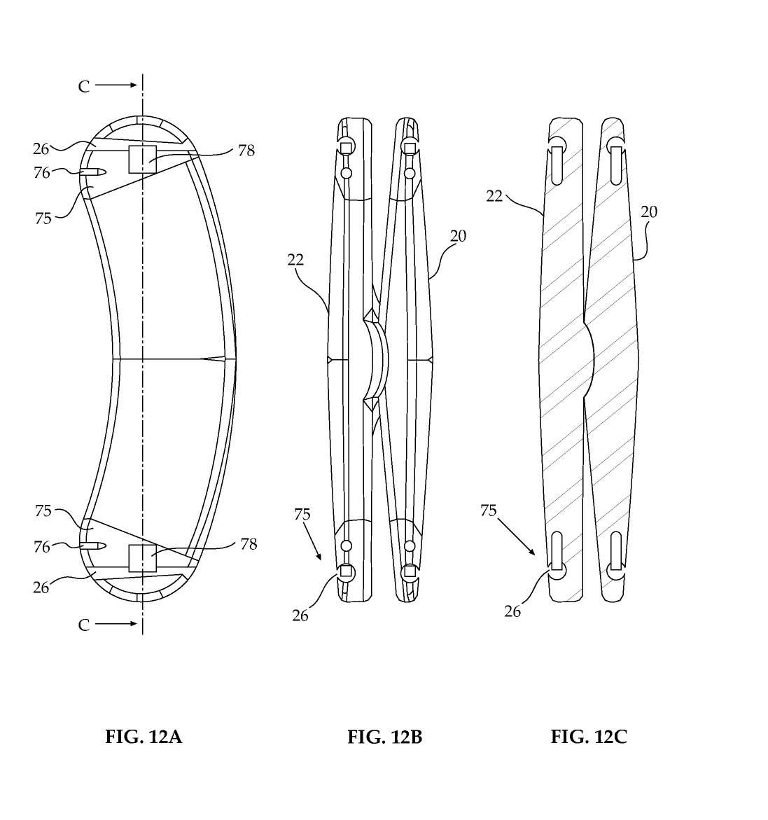

Reference is now made to FIGS. 12A-12C. In accordance with a non-limiting embodiment of the invention, attachment surfaces 20 and 22 are formed with one or more channels 26 and fixation elements 75 are rotatingly disposed in channels 26. Fixation elements 75 are each arranged to pivot about a pivot pin 76 and are each formed with a fixing member 78 (e.g., tab or the like) that protrudes therefrom.

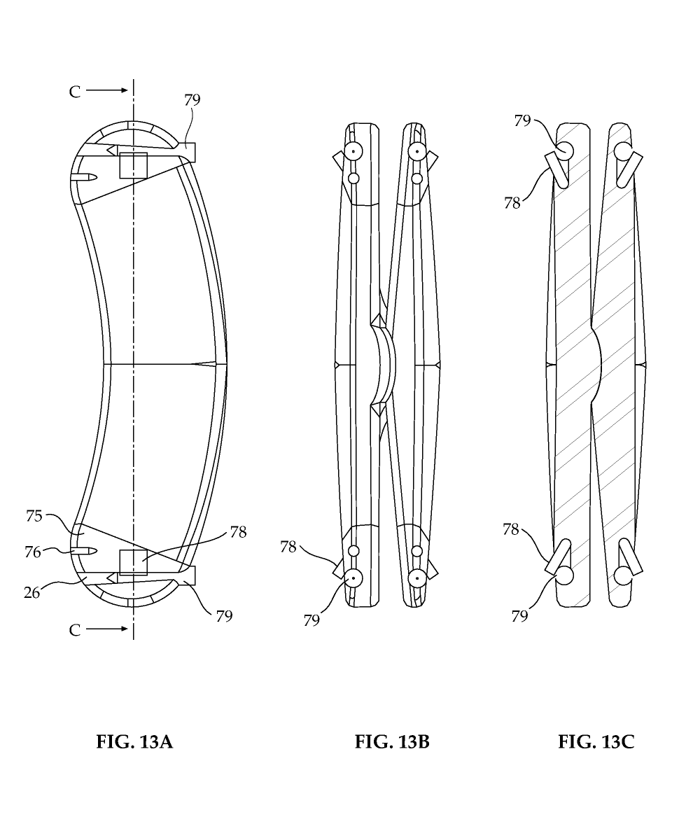

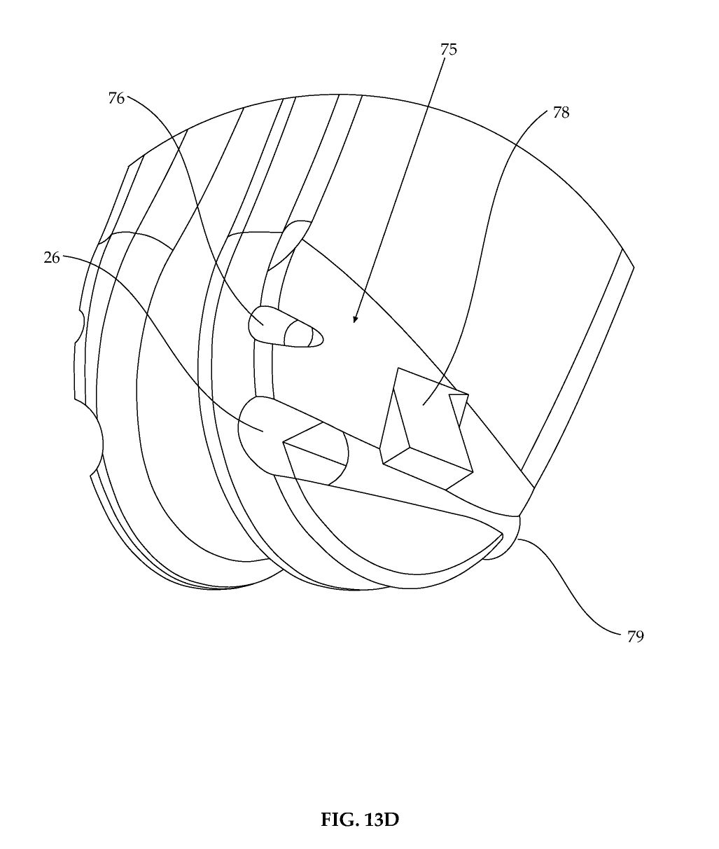

Initially, fixing member 78 sits in channel 26. Referring now to FIGS. 13A-13D, a wedging tool 79 may be inserted into channel 26 which causes fixation element 75 to pivot and forces fixing member 78 out of channel 26. Fixing member 78 is thus deployed to the position shown in FIGS. 13A-13D to press or “bite” into the spinal structure and fix the endoprosthesis into the intervertebral space.

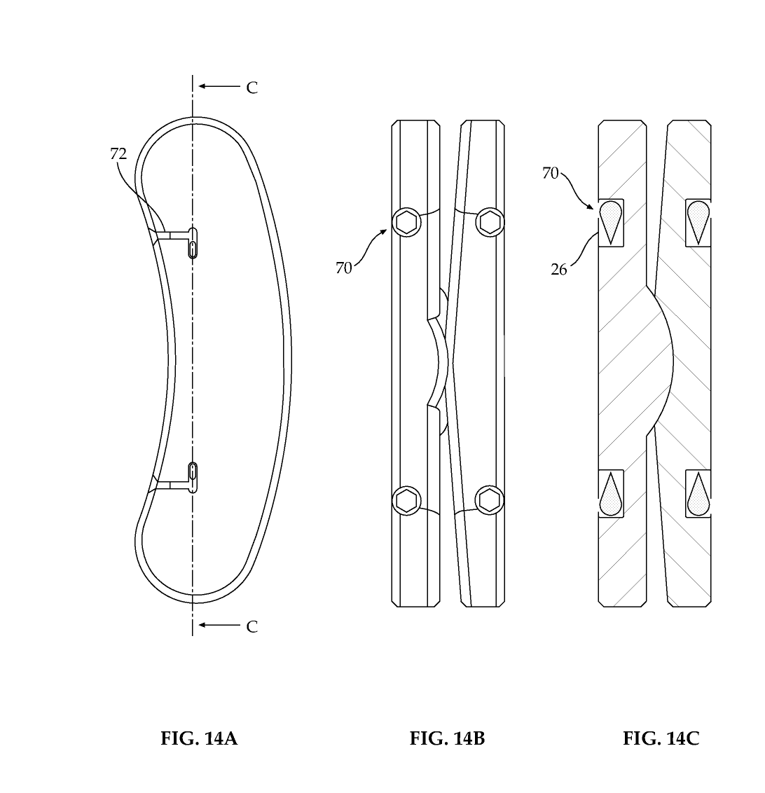

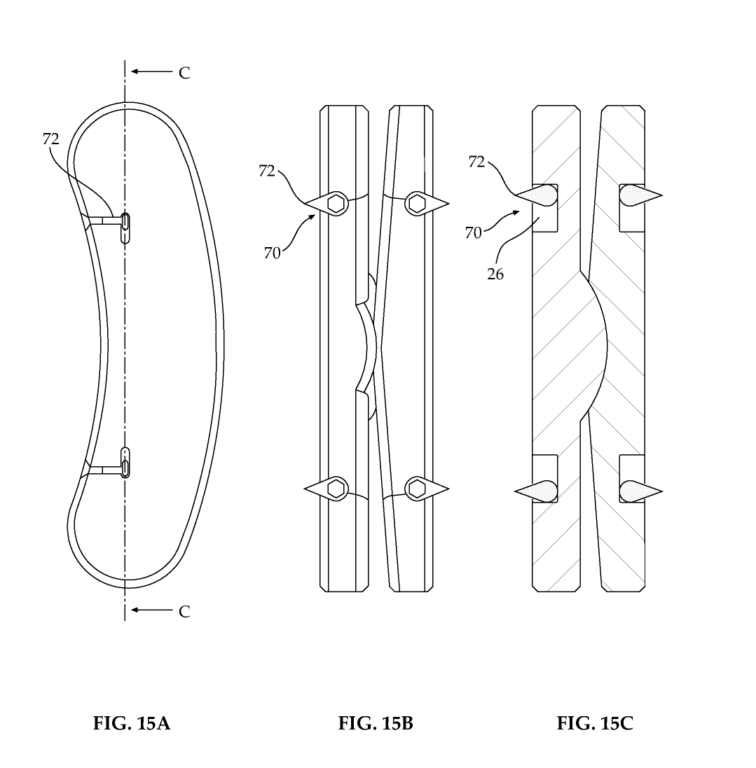

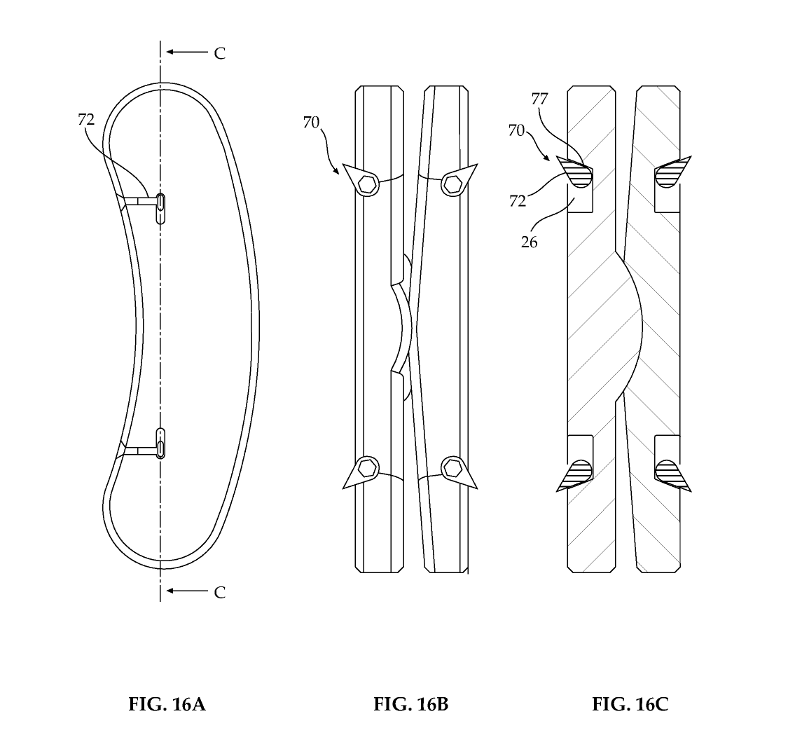

Reference is now made to FIGS. 14A-15C. This embodiment is basically the same as that of FIGS. 10A-11C, with like elements being designated by like reference numerals, except that the channel 26 is shaped differently in the embodiment of FIGS. 14A-15C. FIGS. 16A-16C show a variant of the embodiment of FIGS. 14A-15C, wherein the channel 26 has a terminal end 77, which geometrically locks fixing member 72 of fixation element 70 in place. By “geometric locking” it is meant that a portion of the fixing member 72 moves in the channel, and is prevented from moving further in the channel at least partially by a geometric shape or geometric arrangement of the channel with respect to the fixing member. For example, the terminal end 77 may be angled and/or curved so that fixing member 72 is wedged thereagainst and tends to stay in place.

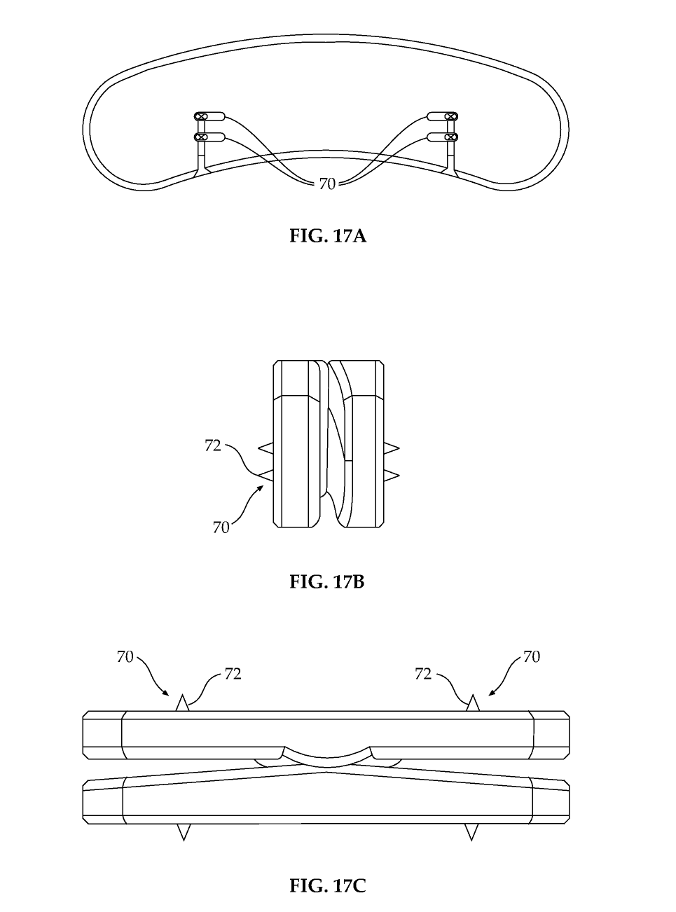

Reference is now made to FIGS. 17A-17C. This embodiment is basically the same as that of FIGS. 10A-11C, except that in the embodiment of FIGS. 17A-17C, fixation elements 70 have a plurality of fixing members 72 formed thereon, and they may be deployed outwards simultaneously by rotating fixation elements 70 as described hereinabove.

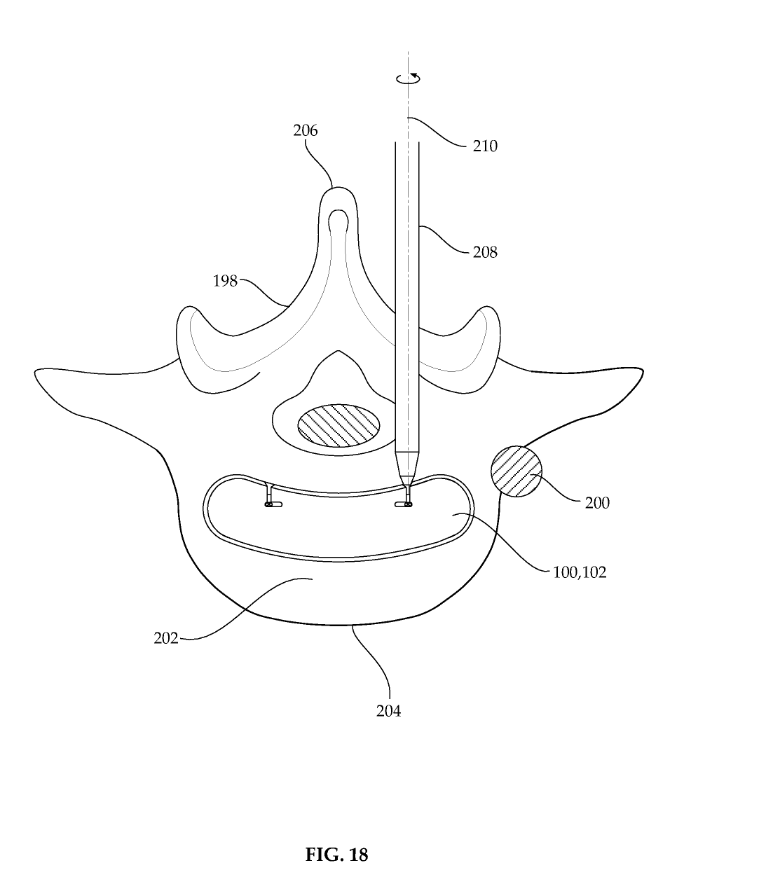

Reference is now made to FIG. 18, which illustrates a top view of a surgical procedure for manipulating the fixation elements, in accordance with an embodiment of the present invention, such as within a typical lumbar vertebra 202. Vertebra 202 includes an anterior portion 204, a posterior portion 206, a spinal cord 198 and a root 200. A manipulating tool 208 may be used for deploying the fixation elements (moving them to a protruding position). Tool 208 may be constructed similar to that of a screwdriver, Allen wrench and the like, depending on the tool interface of the fixation element. Tool 208 may be used in a variety of manipulation procedures, such as but not limited to, TLIF (transforaminal lumbar interbody fusion) or PLIF (posterior lumbar interbody fusion). Tool 208 preferably enters the intervertebral disc space between the spinal cord 198 and the root 200. Rotating tool 208 about its axis of rotation 210 turns the fixation elements and deploys them to protrude outwards for fixing to spinal structure. The fixation elements may be returned to the stowed (generally flush) position by simply turning in the opposite direction with tool 208. Thus tool 208 enables manipulating the protrusion elements with a relatively low risk of injuring spinal tissue, and provides the option for revision surgery, for locating the protrusion elements inside the body of the prosthesis, and for easy removal of the prosthesis.

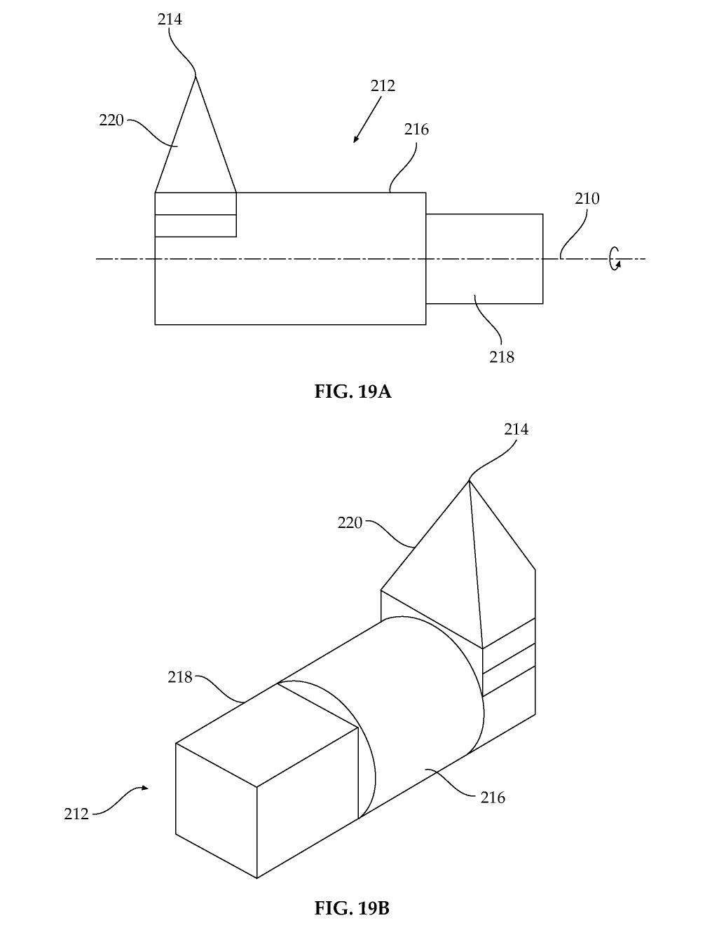

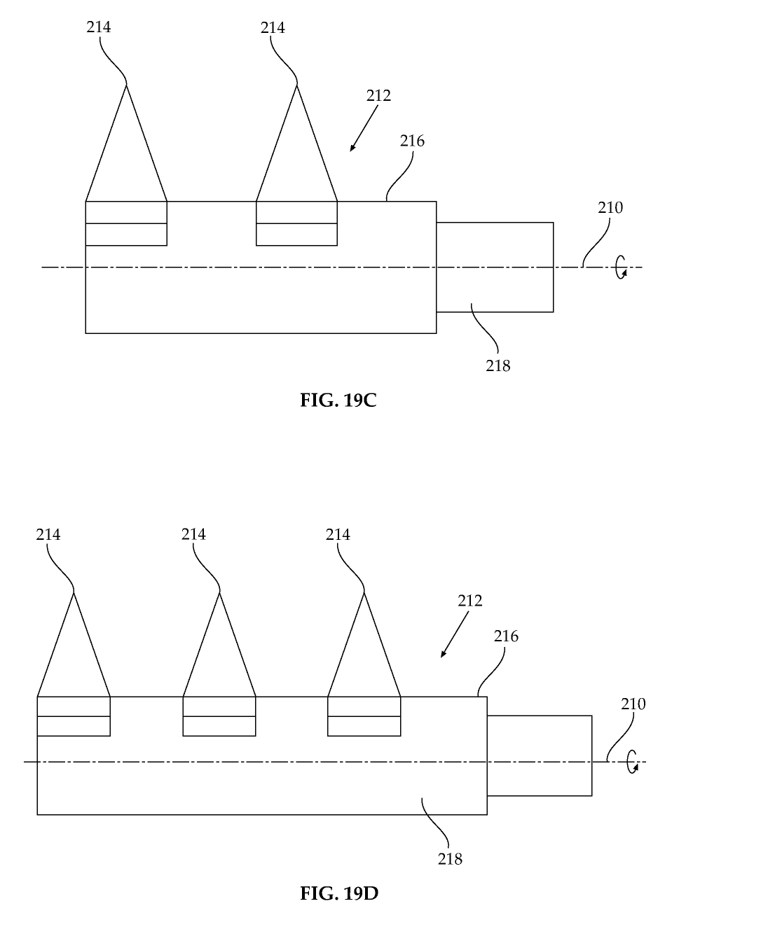

FIGS. 19A, 19B, 19C, 19D, 20A, 20B, 20C, 20D, 21A, 21B, 21C and 22 illustrate further embodiments of initial fixation elements.

FIGS. 19A and 19B illustrate a basic design of a fixation element 212, which has a spike 214. The spike may be pyramid shaped with equal or unequal sides 220. A cylinder shaped body 216 may allow rotation around axis of rotation 210 and has a wrench head 218 for applying torque. Wrench head 218 may be any geometrical head or socket for applying torque, for example hexagonal, square, or slotted.

FIGS. 19C and 19D illustrate other variants of fixation element 212, respectively with two and three spikes 214.

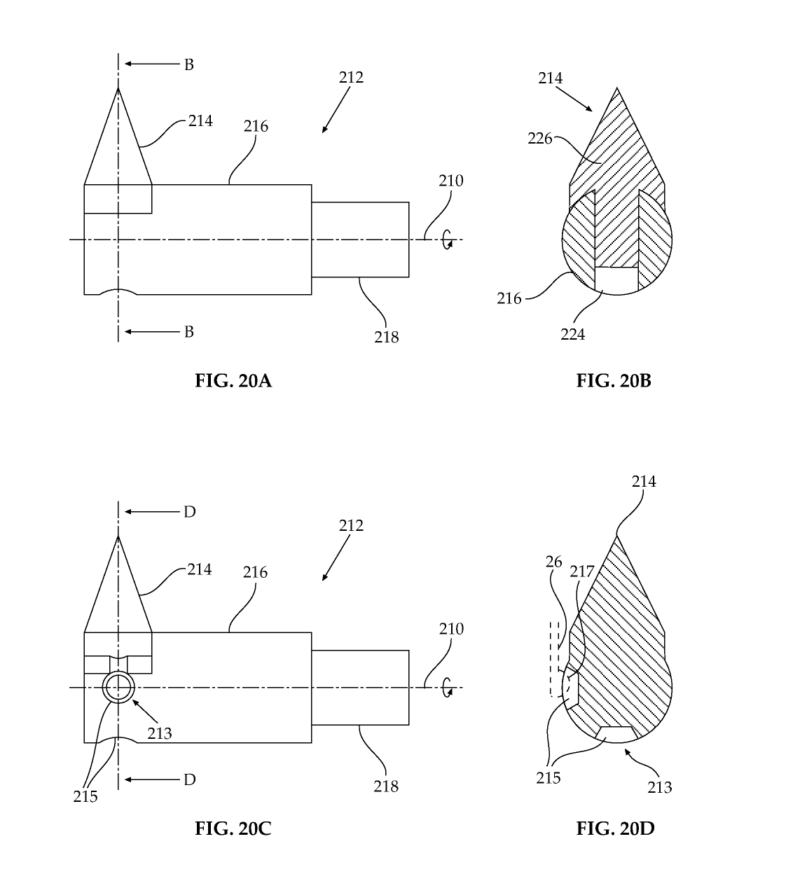

FIGS. 20A and 20B show that fixation element 212 may be manufactured from more than one part. For example, spike 214 may comprise a pointed portion 226 that is inserted in a notch 224 formed in body 216. Pointed portion 226 may be welded or bonded or otherwise joined to body 216.

FIGS. 20C and 20D illustrate a version of fixation element 212, which includes locking members 213 for locking the fixation element 212 in the stowed and deployed positions in the channel 26 in any of the embodiments of the invention, wherein the fixation element is rotated between the stowed and deployed positions in the channel 26. For example, locking members 213 may include detents 215 (e.g., notches or dimples) which “click” into position with lugs 217 (shown in dashed lines in FIG. 20D) found in channel 26.

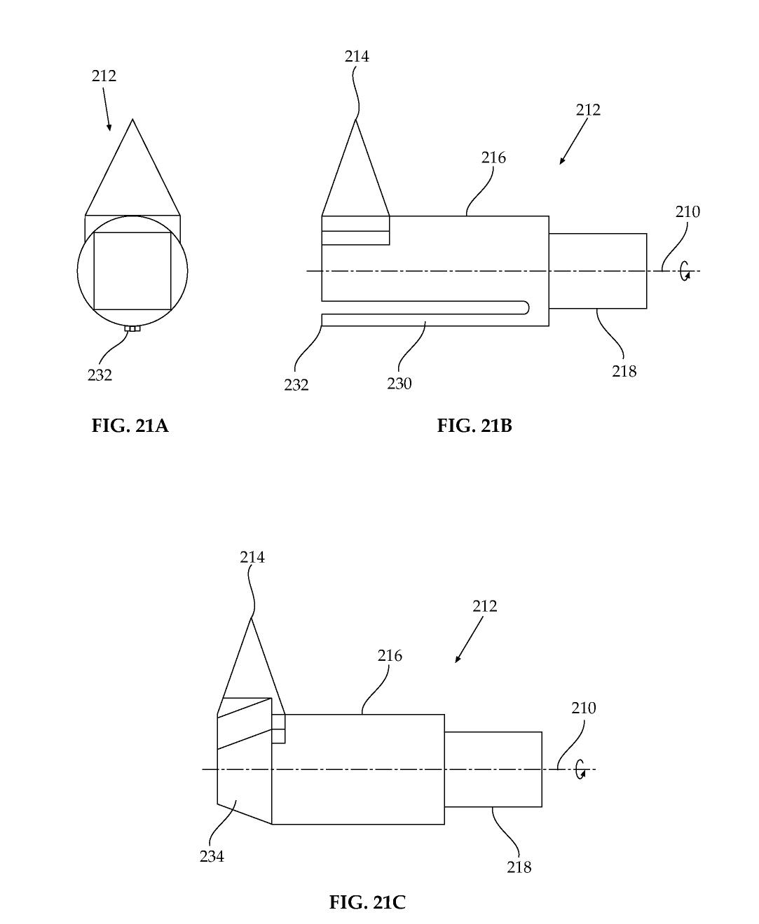

FIGS. 21A and 21B illustrate another kind of locking member for locking the fixation element 212 in the stowed and deployed positions in the channel 26 in any of the embodiments of the invention. In this embodiment, the locking member is a leaf spring 230 and a snap 232 that may snap into place in the channel to keep the fixation element 212 in the desired position.

FIG. 21C illustrates another kind of locking member for locking the fixation element 212 in the stowed and deployed positions. In this embodiment, a chamfered surface 234 may help define positioning of the fixation element 212 and keep it in the desired position.

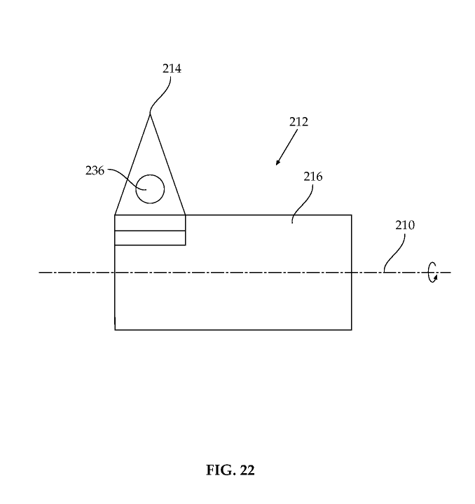

FIG. 22 illustrates another feature, in which the fixation element 212 is formed with a hole 236 in spike 214. Hole 236 may be used to pull or maneuver fixation element 212, for example with a cable or a rigid tool.

Although the invention has been described in conjunction with specific embodiments thereof, it is evident that many alternatives, modifications and variations will be apparent to those skilled in the art. Accordingly, it is intended to embrace all such alternatives, modifications and variations.

What is claimed is:

1. Apparatus comprising:
an endoprosthesis comprising first and second attachment surfaces for attachment to spinal structure; and
a fixation element rotatingly disposed in at least one of said attachment surfaces, said fixation element being rotatable between a stowed position, wherein said fixation element does not substantially protrude from said at least one of said attachment surfaces, and a deployed position, wherein said fixation element does substantially protrude from said at least one of said attachment surfaces for entering and being fixed to the spinal structure.

2. Apparatus according to claim 1, wherein said at least one of said attachment surfaces is formed with a channel and a fixation element is rotatingly disposed in said channel.

3. Apparatus according to claim 1, wherein said fixation element is formed as a rod with a fixing member that protrudes from said rod, wherein in said deployed position, said fixing member protrudes from said at least one of said attachment surfaces for entering and being fixed to the spinal structure.

4. Apparatus according to claim 1, wherein said fixation element comprises a driver interface for turning by means of a manipulating tool.

5. Apparatus according to claim 1, wherein said fixation element pivots about a pivot pin.

6. Apparatus according to claim 2, further comprising a wedging tool for insertion into said channel which causes said fixation element to pivot outwards from said channel.

7. Apparatus according to claim 2, wherein said channel has a terminal end, which geometrically locks said fixation element in place.

8. Apparatus according to claim 1, wherein said fixation element has a plurality of fixing members formed thereon, wherein in said deployed position, said fixing members protrude from said at least one of said attachment surfaces for entering and being fixed to the spinal structure.

9. Apparatus according to claim 1, wherein said fixation element comprises locking members for locking said fixation element in the stowed and deployed positions.

10. Apparatus according to claim 1, wherein said fixation element comprises a fixing member formed with a hole therein, wherein in said deployed position, said fixing member protrudes from said at least one of said attachment surfaces for entering and being fixed to the spinal structure.

11. An endoprosthesis comprising:
a first articulating member formed with a generally concave articulating surface;
and a second articulating member formed with a generally convex articulating surface which articulates with said concave articulating surface, said first and second articulating members each having a generally planar attachment surface for attachment to spinal structure;
a fixation element rotatingly disposed in at least one of said attachment surfaces, said fixation element being rotatable between a stowed position, wherein said fixation element does not substantially protrude from said at least one of said attachment surfaces, and a deployed position, wherein said fixation element does substantially protrude from said at least one of said attachment surfaces for entering and being fixed to the spinal structure.

12. The endoprosthesis according to claim 11, wherein said at least one of said attachment surfaces is formed with a channel and a fixation element is rotatingly disposed in said channel.

13. The endoprosthesis according to claim 11, wherein said fixation element is formed as a rod with a fixing member that protrudes from said rod, wherein in said deployed position, said fixing member protrudes from said at least one of said attachment surfaces for entering and being fixed to the spinal structure.

14. The endoprosthesis according to claim 11, wherein said fixation element comprises a driver interface for turning by means of a manipulating tool.

15. The endoprosthesis according to claim 11, wherein said fixation element pivots about a pivot pin.

16. The endoprosthesis according to claim 12, further comprising a wedging tool for insertion into said channel which causes said fixation element to pivot outwards from said channel.

17. The endoprosthesis according to claim 12, wherein said channel has a terminal end, which geometrically locks said fixation element in place.

18. The endoprosthesis according to claim 11, wherein said fixation element has a plurality of fixing members formed thereon, wherein in said deployed position, said fixing members protrude from said at least one of said attachment surfaces for entering and being fixed to the spinal structure.

19. The endoprosthesis according to claim 11, wherein said fixation element comprises locking members for locking said fixation element in the stowed and deployed positions.

20. The endoprosthesis according to claim 11, wherein said fixation element comprises a fixing member formed with a hole therein, wherein in said deployed position, said fixing member protrudes from said at least one of said attachment surfaces for entering and being fixed to the spinal structure.
